(19) United States
(12) Patent Application Publication
(10) Pub. No.: US 2006/0127381 A1
(43) Pub. Date: Jun. 15, 2006

(54) Live bacterial preparation of bacillus coagulans for the treatment of ulcerative colitis, method for producing the same and use thereof

(76) Inventor: Yunlong Cui, Jiaonan City (CN)

Correspondence Address: WESTMAN CHAMPLIN & KELLY, P.A., SUITE 1400 - INTERNATIONAL CENTRE, 900 SECOND AVENUE SOUTH, MINNEAPOLIS, MN, 55402-3319, US

(21) Appl. No.: 11/191,712

(22) Filed: Jul. 28, 2005

(30) Foreign Application Priority Data
Dec. 15, 2004 (CN) 200410098660.4

(51) Int. Cl. A61K 35/74; C12N 1/20

(52) U.S. Cl. 424/93.46; 435/252.31

(57) ABSTRACT

The present invention discloses a method for preparation of live Bacillus coagulans and the use of the bacteria in treatment of ulcerative colitis. The live bacteria are effective in the treatment of experimental ulcerative colitis in rats. They are also used in the clinical treatment of patients suffering from ulcerative colitis and the efficacy and safety are evaluated. The present invention also discloses the formulation and production method of the live bacterial preparation of Bacillus coagulans, the count of live bacterial in the preparation, and dosage form of the preparation. The present invention shows that the live bacterial preparation is effective and safe for treatment of ulcerative colitis, and the stability of said preparation is good and could be stored at room temperature. Patients can take the preparation for a long term without adverse effect. The present invention provides a new means for the treatment of ulcerative colitis and a new use of live bacterial preparation of Bacillus coagulans in treating ulcerative colitis.

### FIELD OF THE INVENTION

The present invention relates to a live bacterial preparation of Bacillus coagulans for the treatment of ulcerative colitis, method for producing the same and use thereof.

### BACKGROUND OF THE INVENTION

Ulcerative colitis (UC) is a chronic rectal and colonic inflammatory disease with an unknown pathogeny. This disease may take place at any age, mostly 20 to 50 yeas old, and there is no significant difference between man and woman in terms of morbidity rate. UC is quite common in European countries and US with a morbidity of 79˜140/100 thousands of people. The morbidity of UC in Japan is relatively low showing around 3.7-54/100 thousands of people. Although epidemiological survey on UC has not been conducted in China, the morbidity of UC in China is estimated to be not less than that of Japan. In recent years, there is a marked increase in inflammatory bowel disease. The remission rates of mild and severe UC are 70-80% and 50%, respectively. The mortality of fulminant severe UC is even up to 25%. It is of particular concern that a long-term chronic ulcerative colitis would induce canceration and its incidence is 3% to 5%. For patients suffered from UC for more than 20 years, the incidence of canceration could be more than 20%. Although the cause of UC is still unknown, many studies have suggested that UC may be connected with immunity, infection, genetics, intestinal dysbacteriosis, food allergy and psychic factor, etc. Most studies consider the pathological decrease of goblet cell and exhaustive decrease of mucin in intestinal mucosa is one of pathogenic conditions of UC, while factors such as dysbacteriosis, food allergy and psychic factor are important inducing factors.

UC is a recurrent chronic intestinal tract disease with main symptoms of abdominalgia, diarrhea, tenesmus and defecating mucous bloody stool. Young man suffering from UC will bear a long-term social and psychical burden and a life-long treatment. A foreign forward looking study including 496 UC patients shows an annual recurrence rate of 50% after a treatment for 16 months, wherein 1% recurrent patients shows a chronic recurrent course of disease. Currently, the standard therapeutic drug of active stage UC is glucocorticoid steroid hormone, but the recurrence rate and mortality after the treatment are still high. There is no especially effective preparation for the medical treatment of UC at present. Agents such as above adrenalocortical hormone, 5-aminosalicylic acid, salicylazosulfapyridine, metronidazole, rifampicin may remit symptoms, but all of the above agents have a certain degree of side effect, which are insufferable for patient to persist in treatment. The surgical treatment of UC is to excise the whole colon, mostly resulting in sequela. In contrast, microecologic preparation is highly effective for UC in respect that it could regulate flora in intestinal tract and immunity.

We have carried out animal experiments to demonstrate that Bacillus coagulans is quite efficient for treatment of experimental ulcerative colitis in rats. After treatment, the ulcerative area reduces, wet weight of colon decreases, the activity of myeloperoxidase reduces and inflammation is alleviated obviously. For this reason, we conducted an intensive study on the treatment effect of Bacillus coagulans preparation on UC and have developed a new use of live bacteria preparation of Bacillus coagulans in the treatment of UC. This new use will produce a significant socioeconomic performance by relieving the pain of UC patients and safeguarding the health of people.

There is only one Chinese patent concerning Bacillus coagulans, i.e. CN1371620 with a title of “Microecologic Conditioning Agent For Domestic Animal And Fowl”, wherein four species of bacteria including Bacillus coagulans are used to prevent and treat intestinal tract diseases of domestic animal and fowl. For use of Bacillus coagulans in prevention and treatment of human intestinal tract diseases, no patent has been applied. In U.S. Pat. No. 6,723,326, spores (BC) and extracellular products (supernatant and filtrate) of bacillus are topically applied to the skin and mucous membrane of mammal to inhibit the growth of bacteria, fungi and viruses. EP1022023 discloses a composition suitable both as food integrator and for the treatment of intestinal disorders and alterations of the bacterial flora, comprising as active principle a combination of Bacillus coagulans and lysine. WO0954982 discloses compositions for treating gastrointestinal infections, particularly sudden infant death syndrome (SIDS), comprising Bacillus coagulans or extracellular products thereof. WO0134168 discloses compositions comprising a lactic acid-producing bacterial strain, spores or extracellular products produced by the bacterial strains for inhibition of pathogenic bacterial infections. WO0061201 discloses a composition comprising Bacillus coagulans or extracellular products thereof for inhibition of the growth of bacteria on epidermic tissue when using sanitary accessories. In view of above, there is no patent disclosing the use of Bacillus coagulans in the treatment of ulcerative colitis in experimental research or clinical study. For this reason, we especially submit this application.

### SUMMARY OF THE INVENTION

One object of the present invention is to provide a method for treatment of ulcerative colitis, comprising administrating a therapeutically effective amount of live bacteria of Bacillus coagulans to patients in need of such treatment.

In particular, the live bacteria of Bacillus coagulans is a preparation of live bacteria and the Bacillus coagulans is Bacillus coagulans TBC169 (CGMCC No. 1207).

Another object of the present invention is to provide a live bacterial preparation of Bacillus coagulans for the treatment of ulcerative colitis.

A still another object of the present invention is to provide a method for producing a live bacteria powder of Bacillus coagulans.

The inventor performs experimental studies for treatment of ulcerative colitis with a known Bacillus coagulans strain and a new screened Bacillus coagulans strain, and find both Bacillus coagulans preparation including live bacteria powder of Bacillus coagulans has a good treatment effect for ulcerative colitis. However, the new screened Bacillus coagulans strain produce a better treatment effect than that of known Bacillus coagulans strain. The present invention is thus accomplished based on the above finding.

The producing method of live bacteria preparation of Bacillus coagulans for the treatment of ulcerative colitis comprises following steps: incubating Bacillus coagulans by fermentation; drying bacteria slurry after centrifugation to obtain live bacteria powder, which is then prepared as suitable dosage forms. Ulcerative colitis is characterized in that pathological change mostly occurs at rectum and sigmoid flexure, or throughout the whole colon. A microscopic examination of colon can show mucosal blooding, edema, erosion or superficial ulcertion on affected intestine with mucus and inflammatory exudate covered thereon. Bleeding, inflammatory cell infiltration, epithelial cell decreasing, crypt collapsing and distorting, abscess formation are also often found in microscopic examination. Chronic ulcer may heal to form scar or pseudopolyp. Experimental study is to administrate dextran sodium sulfate (DSS) to SD rat by i.g. and rectal injection to induce experimental ulcerative colitis model, and then subject the modeled rat to experimental treatment with a live bacterial tablet of Bacillus coagulans, whereby the treatment effect of tablet is evaluated. The use is to apply the live bacterial tablet of Bacillus coagulans to clinically treat patients suffering from ulcerative colitis, whereby the efficacy and safety of the live bacterial preparation for UC patients is evaluated.

The term “dosage form” used herein refers to preparing the medicine in a certain dosage form (solid or liquid dosage form) and applying the medicine as a preparation for the treatment or prevention. The efficacy, safety, rationality and accuracy of preparation reflect the quality of medicine and determine the effect of administration.

All of percents or ratios used herein are based on weight or weight/volume, or volume/weight, unless otherwise indicated. In addition, all the measurements in this context are performed at 25° C., unless otherwise indicated.

### DETAILED DESCRIPTION OF THE INVENTION

The present inventor has developed preparations (such as a tablet) comprising live bacteria powder of Bacillus coagulans and suitable adjuvants. The animal research and clinical study has shown the preparations have good therapeutic effect of microecologic probiotic on human intestinal tract diseases (such as diarrhea). Thus the present invention has solved a stubborn problem in the medical field. The present invention will be described in detail in the following.

The present invention provides a use of live bacteria of Bacillus coagulans in the preparation of medicaments for the treatment of ulcerative colitis, wherein the live bacteria of Bacillus coagulans is a preparation of live bacteria. The Bacillus coagulans may be any Bacillus coagulans strains, and includes, but is not limited to Bacillus coagulans TBC169 and Bacillus coagulans, Hammer, 1915. The inventor has selected a new Bacillus coagulans strain which could produce an excellent effect in present invention. The new Bacillus coagulans strain is named as Bacillus coagulans TBC169 and has been deposited with an accession number of CGMCC 1207 at China General Microbiological Culture Collection Center (CGMCC), Beijing, China on Aug. 23, 2004.

The present invention also provides a live bacterial preparation of Bacillus coagulans for the treatment of ulcerative colitis, comprising live bacteria powder of Bacillus coagulans and a pharmaceutically acceptable carrier. The Bacillus coagulans is Bacillus coagulans TBC169 (CGMCC No. 1207). Said pharmaceutically acceptable carrier may be any well-known pharmaceutically acceptable carrier, in particularly microcrystalline cellulose, mannitol, glucose, defatted milk powder, polyvinylpyrrolidone and starch, or mixture thereof.

The live bacteria powder of Bacillus coagulans can be prepared from fermentation product by various known methods, and preferably by centrifuging liquid fermentation product of Bacillus coagulans and drying resultant wet bacteria slurry, wherein the drying is accomplished by any drying method as long as it can keep the activity of Bacillus coagulans. Preferably, the drying is selected from the group consisted of freeze drying, spray drying, heat drying or combination thereof.

Preferably, the live bacterial count of Bacillus coagulans in the preparation is no less than 1.0×10⁶ cfu/g, based on the total weight of the preparation.

In a preferable embodiment of the present invention, the preparation comprises following components based on the total weight of the preparation:

| | |
|---|---|
| live bacteria powder of Bacillus coagulans | 0.05% to 80% |
| microcrystalline cellulose | 0% to 90% |
| mannitol | 0% to 90% |
| polyvinylpyrrolidone | 0% to 90% |
| glucose | 0% to 90% |
| defatted milk powder | 0% to 90% |
| starch | 0% to 90% |

wherein the contents of said microcrystalline cellulose, mannitol, polyvinylpyrrolidone, glucose, defatted milk powder, and starch can not be 0% at the same time.

In another preferable embodiment of the present invention, the preparation comprises following components based on the total weight of the preparation:

| | |
|---|---|
| live bacteria powder of Bacillus coagulans | 0.05% to 70% |
| microcrystalline cellulose | 20% to 90% |
| mannitol | 5% to 90% |
| polyvinylpyrrolidone | 5% to 90%. |

In still another preferable embodiment of the present invention, the preparation comprises following components based on the total weight of the preparation:

| | |
|---|---|
| live bacteria powder of Bacillus coagulans | 0.05% to 80% |
| microcrystalline cellulose | 10% to 70% |
| glucose | 10% to 90%. |

In still another preferable embodiment of the present invention, the preparation comprises following components based on the total weight of the preparation:

| | |
|---|---|
| live bacteria powder of Bacillus coagulans | 0.05% to 70% |
| glucose | 10% to 90% |
| defatted milk powder | 10% to 90% |
| starch | 10% to 90%. |

Preferably, the preparation is in a form of tablet, capsule, powder, or granule.

The present invention further provides a method for producing above live bacterial preparation of Bacillus coagulans for the treatment of ulcerative colitis, comprising following steps:

1) inoculating Bacillus coagulans in a liquid medium and performing multistage amplification incubation;

2) centrifuging the liquid culture of step 1) and collecting a wet bacteria slurry, which is then subjected to drying, pulverizing, and obtaining a dry bacteria powder;

3) mixing the dry bacteria powder of step 2) with a pharmaceutically acceptable carrier to produce the final preparation form.

Preferably, the multistage amplification incubation is a three-stage amplification incubation.

Preferably, the incubation temperature of the multistage amplification incubation is between 30° C. and 55° C., and the incubation period is 6-72 hours.

Preferably, the seed of first-stage culture of said multistage amplification incubation is obtained according to the following method: dissolving solid Bacillus coagulans in physiological saline, wherein the weight ratio of Bacillus coagulans to physiological saline is 1:10 to 1:100; activating in a water bath at 50-80° C. for 5-15 minutes.

The new Bacillus coagulans strain TBC169 screened by the present inventor possesses the following features: Bacillus coagulans TBC169 is a facultative anaerobe, and its colony on casitone media (agar) is off-white or cream white with an uneven edge; Gram-positive, rod shape, oval spore in the center or at one end of most thallus, nonexpansion; capable of making use of glucose, arabinose, xylose, mannose for fermentation; incapable of hydrolyzing starch; positive reactions for catalase, indole and V-P; negative for nitrate reducing reaction; capable of coagulating milk. The optimal pH range for Bacillus coagulans TBC 169 is between 30° C. to 55° C. Spores of the strain can tolerate pasteurization, and can be stored at room temperature with an excellent stability.

### EXAMPLES

The following examples relating to this invention are illustrative and should not be construed as specifically limiting the invention.

### Example 1

### Preparation of Bacteria Powder of Bacillus coagulans

Bacillus coagulans strain in a tube was dissolved in a 100 ml sterilized Erlenmeyer flask filled with 10 ml of physiological saline and suitable amount of glass bead. After activation for 10 minutes, 1 ml bacteria suspension was inoculated into a 250 ml Erlenmeyer flask filled with 50 ml of amplification media by 1 ml sterile pipette, and incubation was continued in shaking incubator (190 rpm) at 37° C. for 24 hours. The culture was transferred to a 2500 ml baffled Erlenmeyer flask filled with 450 ml of amplification media and incubated with shaking at 37° C. for 24 hours. After microscopic examination for contamination, the culture was again transferred to a seeding tank filled with 4.5 L amplification media and subjected to aerobic culture (air inflation amount 3:1) for 24 hours. After microscopic examination for contamination, the culture was then transferred to a fermentation tank filled with 45 L amplification media and subjected to aerobic culture (air inflation amount 3:1) for 24 hours. The incubation was finished when sporulation reaching 80%, and centrifuged at 12000 rpm with continuous centrifuge. The collected wet bacteria slurry was weighted, suitable amount of defatted milk powder was added at a ratio of 1:1 (w/v), dried, pulverized and kept at room temperature ready for use.

### Example 2

### Preparation of Live Bacteria Tablet and Capsule of Bacillus coagulans

According to the count of live bacteria in bacteria powder of Bacillus coagulans, microcrystalline cellulose, mannitol and polyvinylpyrrolidone were added in following proportion to keep the count of live bacteria no less than 1.0×10⁶ cfu/g. Preparation process could be found in “Tablet and Capsule”, Practical Pharmaceutical Preparation Technology, pages 1-30, 1^{st} ed., 1999, the peoples medical publishing house, which is incorporated herein by reference in its entirety.

The following is a formulation of live bacteria tablet of Bacillus coagulans:

| Components | % (weight) | |
|---|---|---|
| Live bacteria powder of Bacillus coagulans | 1.00 | part |
| microcrystalline cellulose | 55.00 | parts |
| mannitol | 25.00 | parts |
| polyvinylpyrrolidone | 19.00 | parts |

Above components 1-4 were stirred well to be homogenous, and then compressed into tablets in unit dosage according to conventional tabletting technology.

The following is a formulation of live bacteria capsule of Bacillus coagulans:

| Components | % (weight) |
|---|---|
| Live bacteria powder of Bacillus coagulans | 15.00 parts |
| microcrystalline cellulose | 25.00 parts |
| glucose | 60.00 parts |

Above components 1-3 were stirred well to be homogenous, and then made into capsules in unit dosage according to conventional encapsulating technology.

### Example 3

### Preparation of Live Bacteria Medicinal Powder of Bacillus coagulans

According to the count of live bacteria in bacteria powder of Bacillus coagulans, defatted milk powder, glucose and starch were added in following proportion to keep the count of live bacteria no less than 1.0×10⁶ cfu/g. Preparation process could be found in “Medicinal Powder”, Practical Pharmaceutical Preparation Technology, pages 1-30, 1^{st} ed., 1999, the peoples medical publishing house, which is incorporated herein by reference in its entirety.

The following is a formulation of live bacteria medicinal powder of Bacillus coagulans:

| Components | % (weight) |
|---|---|
| Live bacteria powder of Bacillus coagulans | 20.00 parts |
| defatted milk powder | 30.00 parts |
| glucose | 30.00 parts |
| starch | 20.00 parts |

Above components 1-4 were stirred well to be homogenous, and then made into bagged medicinal powder in unit dosage according to conventional process.

The preparation of live bacteria granule of Bacillus coagulans was similar to that of powder, and was omitted herein.

Preparation and Treatment of Experimental Ulcerative Colitis Model in Rat

60 rats were kept under a sterile environment before test. Normal intestinal flora was taken from stools in the rats' anuses and incubated. 10 rats were taken as normal control and other 50 rats as experimental group. According a modified UC rat modeling method (Xiaoping Wu etc., Chinese Journal of Digestion, Vol. 23(5):305, 2003), 3% dextran sodium sulfate (DSS) solution was i.g. administrated to rats with empty stomachs (1 ml/100 g), once a day for 7 days. At the last time, 1 ml/rat of DSS was additionally injected into recta through anuses. After 7 days, stools were taken for flora analysis. 10 rats were then sacrificed and colons were removed by laparocolectomy. The wet weights of colons were measured (g/100 g body weight). The colons was fixed with formaldehyde and stained with alcian blue solution (Sino-American Biotec). The point counts of ulcer and erosion parts were observed by naked eye. Areas (cm²) of the largest stained blue points were measured with calipers. After the confirmation of UC formation, rats were divided into 4 groups, 10 rats per group. The rats were administrated i.g. with live bacteria tablet of Bacillus coagulans(BC) (10⁷ cfU/ml, 10⁶ cfU/ml), 0.02 g/ml salicylazosulfapyridine (SASP) and normal saline solution (NS), respectively, twice a day for 21 days, afterwhich the stools were taken for flora analysis. Thereafter, all of the rats were sacrificed. Colon specimens were treated according to above method and corresponding indicators were determined. In addition, intestinal tissue masses were sampled to prepare 5% homogenates, which were determined for their activities of myeloperoxidase (MPO) (U/g·wet slice) with myeloperoxidase test kit (Nanjing JianCheng Bioengineering Institute) by ultraviolet spectrometry (Beijing Technical Application Institute).

The intestinal flora was incubated and analyzed as follows. Fresh stools were extruded out from rats' anuses under sterile condition before or after modeling and after treatment, respectively. The stools were subjected to a 10 times serial dilution and 0.1 ml of diluted homogenate solutions of 10⁻², 10⁻³, 10⁻⁴ of initial concentration were inoculated and spread evenly on plates with suitable media. The aerobic and anaerobic bacteria were incubated at 37° C. for 48 hours and 72 hours, respectively. Counts of live bacteria were observed by naked eye. Treatment results were as follows. After the administration of DSS by i.g. and rectal injection, typical experimental ulcerative colitis was developed. After treating with BC, SASP and NS for 21 days, body weights of rats in each groups increased, wherein rats treated with 10⁷ cfu/ml of BC gained in weight significantly (p<0.05). Rats in treatment groups were statistically significant in terms of wet weight of colon, intestinal ulcer, and/or erosion point number, area, MPO activity, compared with modeling group or modeling group treated with normal saline solution (p<0.05 or p<0.01). This showed that BC tablet had significant therapeutic effect on experimental UC in rat, which provided a scientific basis for clinical administration. As for the incubation and analysis of intestinal flora, the numbers of live bifidobacteria in UC modeling-treated groups decrease obviously, and increase significantly after the treatment (p<0.01). Other florae, however, have no significant change (P>0.05). B. coagulans was colonized in the intestinal tract.

Clinical Study of Treatment of Ulcerative Colitis with live bacteria tablet of Bacillus coagulans

The study method was as follows.
Case selection. According to the diagnostic standard of ulcerative colitis in “Gastroenterology” edited by Zhitian Zheng, patients suffering from intestinal malignant diseases or with apparent bleeding, perforation, diarrhea, obstruction were excluded from subjects. More than 400 patients satisfying above diagnostic standard were taken as subjects. Informed consents were signed by patients.
Treatment method. Subjects were randomly divided into an administration group and a control group, wherein said administration group included 300 patients and live bacteria tablets of Bacillus coagulans were administrated orally three times a day, 6 tablets per time, while the control group included 100 patients and tablets of golden bifidobacterium (Golden Bifid, Inner Mongolia ShuangQi Pharmaceutic Co. Ltd.) were administrated orally three times a day, 4-6 tablets per time, for a period of treatment of 21-30 days. Clinical test had been conducted at 10 national clinic bases.
Items for evaluation of efficacy. The therapeutic effects were evaluated, based on the improvements of symptoms such as diarrhea, abdominalgia, tenesmus and defecating mucous bloody stool and the decreases of focus areas before and after the treatment.
Criteria for evaluation of efficacy. Clinical cure: the area of ulcer disappears completely; slight degree of rubefaction without obvious edema; subjective symptoms disappear completely or substantially. Apparently effective: the area of ulcer disappears substantially; however, still with inflammation and most of the symptoms disappear. Effective: the focus area of ulcer decreased by more than 50% and most of the symptoms were reduced significantly. Ineffective: the focus area of ulcer decreased by less than 50% without improvements in respect of subjective symptoms.
Safety evaluation. The safety evaluation was performed on the basis of side effect and abnormal assessments for related examination such as blood picture and liver-kidney function examination after the administration to patient.
   Evaluation of Therapeutic Effect on Experimental Ulcerative Colitis of Rat

The treatment method was same as the above and therefore omitted here.

Treatment results showed that wet weights of colons of UC rats after treatment reduced obviously, and counts of ulcer point and focus area decreased significantly. In addition, MPO activity reduced significantly and inflammation ameliorated obviously or disappeared. In view of above, the therapeutic effect was very significant (see Table 1).

TABLE 1
DSS modeling and treatment of experimental colitis of rat (x ± S.D)

| | | | | | | Ulcer or | | |
|---|---|---|---|---|---|---|---|---|
| | | | | | Intestinal | erosion | | MPO |
| | | Body weight (g) | | | wet weight | points | | activity |
| | | Before | After | After | (g/100 g | Count of | area | (U/g wet |
| Group | Rat | modeling | modeling | treatment | body weight) | Points | (cm²) | weight) |
| Normal | 10 | 88.0 ± 9.20 | 134.00 ± 12.70 | 292.00 ± 14.00 | 0.50 ± 0.04 | 0 | 0 | 0.07 ± 0.05 |
| group | | | | | | | | |
| Modeling | 10 | 90.00 ± 8.16 | 127.00 ± 8.23 | N.D. | 1.26 ± 0.08 | 4.20 ± 0.92 | 0.68 ± 0.21 | 1.33 ± 0.31 |
| goup | | | | | | | | |
| Modeling + | 10 | 86.00 ± 8.43 | 130.00 ± 6.70 | 255.00 ± 16.50 | 0.61 ± 0.07** | 3.50 ± 0.97 | 0.42 ± 0.16** | 0.68 ± 0.35 |
| Normal | | | | | | | | |
| saline | | | | | | | | |
| Modeling + | 10 | 89.00 ± 9.94 | 127.00 ± 9.50 | 275.00 ± 22.70^{Δ} | 0.48 ± 0.05**^{ΔΔ} | 1.80 ± 1.23**^{ΔΔ} | 0.14 ± 0.14**^{ΔΔ} | 0.20 ± 0.10**^{ΔΔ} |
| BC 10⁷ | | | | | | | | |
| Modeling + | 10 | 93.00 ± 9.49 | 133.00 ± 9.50 | 267.00 ± 16.70 | 0.50 ± 0.05**^{ΔΔ} | 1.60 ± 1.07**^{ΔΔ} | 0.15 ± 0.10**^{ΔΔ} | 0.29 ± 0.29*^{ΔΔ} |
| BC 10⁶ | | | | | | | | |
| Modeling + | 10 | 86.00 ± 8.43 | 131.00 ± 8.80 | 264.00 ± 13.50 | 0.54 ± 0.05**^{Δ} | 2.20 ± 0.79**^{ΔΔ} | 0.22 ± 0.18**^{Δ} | 0.20 ± 0.10**^{ΔΔ} |
| SASP | | | | | | | | |

Compared with modeling group, *p < 0.05, **p < 0.01, compared with modeling group treated with Normal saline, ^{Δ}p < 0.05, ^{ΔΔ}p < 0.01.

Comparison of Two Strains of Bacillus coagulans in Terms of Treatment Effect on UC Rat

Treatment method: (1) The modeling of UC rat and evaluated items and method were same as the above. (2) Administration method. Three groups of rats were i.g. administrated with 10⁷ cfu/ml live bacteria solutions of Bacillus coagulans TBC169, Bacillus coagulans(a Japan strain, isolated from “preparation of lactic acid bacteria with spore” produced by Japan Sankyo Co., Ltd, and normal saline, respectively, 10 UC rats a group, twice a day for 21 days.

Treatment results showed that wet weights of colons of UC rats in 2 treatment groups reduced, and counts of ulcer point and focus area decreased significantly after treatment. In addition, MPO activity also reduced. There was a significant difference between the two treatment groups and normal saline group (p<0.05 or p<0.01) (see Table 2).

Although there was no significant difference between the two treatment groups, each parameter in TBC169 strain group was lower than that of Japan strain group and showed a better therapeutic effect.

In summary, these results demonstrated TBC169 strain was better than the Japan strain and showed an excellent therapeutic effect (see Table 2).

TABLE 2
Comparison of two strains of Bacillus coagulans in the
terms of treatment effect on UC rat (x ± S.D)

| | | Intestinal wet | Count of ulcer | | MPO activity |
|---|---|---|---|---|---|
| | | weight (g/100 g | points | Area of ulcer | (u/g wet |
| Group | Rat | body weight) | (points) | (cm²) | weight) |
| Modeling + | 10 | 0.65 ± 0.08 | 3.80 ± 0.98 | 0.45 ± 0.17 | 0.70 ± 0.37 |
| Normal saline | | | | | |
| Modeling + | 10 | 0.46 ± 0.03** | 1.72 ± 0.90** | 0.13 ± 0.10** | 0.18 ± 0.09** |
| TBC169 | | | | | |
| Modeling + | 10 | 0.49 ± 0.07** | 1.92 ± 1.12** | 0.15 ± 0.14** | 0.24 ± 0.12** |
| BC (Japan strain) | | | | | |

Compared with normal saline group, **P < 0.01

1. A method for treatment of ulcerative colitis, comprising administrating a therapeutically effective amount of live bacteria of Bacillus coagulans to subjects in need of such treatment.

2. The method according to claim 1, wherein said live bacteria of Bacillus coagulans is a preparation of live bacteria.

3. The method according to claim 1, wherein said Bacillus coagulans is Bacillus coagulans TBC169 CGMCC No. 1207.

4. A live bacterial preparation of Bacillus coagulans for the treatment of ulcerative colitis, comprising live bacteria powder of Bacillus coagulans and a pharmaceutically acceptable carrier.

5. The preparation according to claim 4, wherein said Bacillus coagulans is Bacillus coagulans TBC169 CGMCC No. 1207.

6. The preparation according to claim 4, wherein the pharmaceutically acceptable carrier is one or more selected from the group consisted of microcrystalline cellulose, mannitol, glucose, defatted milk powder, polyvinylpyrrolidone and starch, or mixture thereof.

7. The preparation according to claim 4, wherein the live bacteria powder of Bacillus coagulans is prepared by centrifuging liquid fermentation product of Bacillus coagulans and drying resultant wet bacteria slurry, wherein the drying is accomplished by freeze drying, spray drying, heat drying or combination thereof.

8. The preparation according to claim 7, wherein the live bacterial amount of Bacillus coagulans of the preparation is no less than 1.0×10⁶ cfu/g, based on the total weight of the preparation.

9. The preparation according to claim 4, wherein said preparation comprises following components based on the total weight of the preparation:

| | |
|---|---|
| live bacteria powder of Bacillus coagulans | 0.05% to 80% |
| microcrystalline cellulose | 0% to 90% |
| mannitol | 0% to 90% |
| polyvinylpyrrolidone | 0% to 90% |
| glucose | 0% to 90% |
| defatted milk powder | 0% to 90% |
| starch | 0% to 90% |

wherein the contents of the microcrystalline cellulose, mannitol, polyvinylpyrrolidone, glucose, defatted milk powder, and starch are not 0% at the same time.

10. The preparation according to claim 9, wherein the preparation comprises following components based on the total weight of the preparation:

| | |
|---|---|
| live bacteria powder of Bacillus coagulans | 0.05% to 70% |
| microcrystalline cellulose | 20% to 90% |
| mannitol | 5% to 90% |
| polyvinylpyrrolidone | 5% to 90%. |

11. The preparation according to claim 9, wherein the preparation comprises following components based on the total weight of the preparation:

| | |
|---|---|
| live bacteria powder of Bacillus coagulans | 0.05% to 80% |
| microcrystalline cellulose | 10% to 70% |
| glucose | 10% to 90%. |

12. The preparation according to claim 9, wherein the preparation comprises following components based on the total weight of the preparation:

| | |
|---|---|
| live bacteria powder of Bacillus coagulans | 0.05% to 70% |
| glucose | 10% to 90% |
| defatted milk powder | 10% to 90% |
| starch | 10% to 90%. |

13. The preparation according to claim 9, wherein the preparation is in a form of tablet, capsule, powder, or granule.

14. A method for producing the preparation according to claim 4, comprising following steps:
1) inoculating Bacillus coagulans in a liquid medium and performing multistage amplification incubation;
2) centrifuging the liquid culture of step 1) and collecting a wet bacteria slurry, which is then subjected to drying, pulverizing, and obtaining a dry bacteria powder;
3) mixing the dry bacteria powder of step 2) with a pharmaceutically acceptable carrier to produce the final preparation form.

15. The method according to claim 14, wherein the multistage amplification incubation is three-stage amplification incubation.

16. The method according to claim 15, wherein the incubation temperature of the multistage amplification incubation is between 30° C. and 55° C., and the incubation period of each stage is 6-72 hours.

17. The method according to claim 16, wherein the seed of first-stage culture of multistage amplification incubation is obtained according to following method: dissolving solid Bacillus coagulans in physiological saline, wherein the weight ratio of Bacillus coagulans to physiological saline is 1:10 to 1:100; activating in a water bath at 50-80° C. for 5-15 minutes.
